(19) United States
(12) Patent Application Publication
(10) Pub. No.: US 2021/0023355 A1
(43) Pub. Date: Jan. 28, 2021

(54) METHOD OF PRODUCING MICRONEEDLE ARRAY, MICRONEEDLE ARRAY, AND MICRONEEDLE ARRAY UNIT

(71) Applicant: FUJIFILM Corporation, Tokyo (JP)

(72) Inventor: Kenichiro TAMAKI, Kanagawa (JP)

(73) Assignee: FUJIFILM Corporation, Tokyo (JP)

(21) Appl. No.: 16/910,067

(22) Filed: Jun. 24, 2020

(30) Foreign Application Priority Data
Jul. 26, 2019 (JP) 2019-137873

(51) Int. Cl. A61M 37/00

(52) U.S. Cl. CPC A61M 37/0015; A61M 2037/0023; A61M 2037/0053

(57) ABSTRACT

The present invention provides a method of producing a microneedle array with excellent mountability on a container and excellent handleability such as puncture or disposal, a microneedle array, and a microneedle array unit.

Provided are a method of producing a microneedle array including a step of placing a support member which includes a disk portion having a through-hole and a columnar portion formed on a first surface of the disk portion, on a pattern surface of a mold having needle-like depressions, a step of supplying a base material liquid from a side of the first surface of the disk portion and filling the needle-like depressions with the base material liquid, and a drying step of drying the base material liquid to integrally mold a base material layer and the support member; the produced microneedle array; and a microneedle array unit including the microneedle array and a container.

### CROSS-REFERENCE TO RELATED APPLICATIONS

The present application claims priority under 35 U.S.C. § 119 to Japanese Patent Application No. 2019-137873 filed on Jul. 26, 2019. The above application is hereby expressly incorporated by reference, in its entirety, into the present application.

### BACKGROUND OF THE INVENTION

### 1. Field of the Invention

The present invention relates to a method of producing a microneedle array, a microneedle array, and a microneedle array unit.

### 2. Description of the Related Art

In recent years, a microneedle array (micro-needle array) has been known as a new dosage form that enables administration of drugs such as insulin, vaccines, and human growth hormones (hGH) into the skin without pain. The microneedle array is formed such that biodegradable microneedles containing a drug are arranged in an array. Each microneedle is pierced into the skin by attaching this microneedle array to the skin, and these microneedles are absorbed in the skin so that the drug contained in each microneedle is administered into the skin.

In order to easily puncture the microneedle array into the skin, a container (also referred to as an applicator) that presses the microneedle array against the skin is used, or a support is provided on a side of the rear surface of the microneedle array.

For example, JP2019-013524A describes a microneedle in which a substrate and a projection are integrally molded and which comprises a protrusion or a depression on a side of a surface of the substrate which comprises the projection in order to easily mount the needle on an applicator. WO2010/140401A describes a microneedle array formed by bringing a porous support into contact with the surface of a base material liquid before a step of drying the base material liquid and impregnating the porous support with the base material liquid so that the support is integrated with the base material liquid.

### SUMMARY OF THE INVENTION

However, the microneedle array described in JP2019-013524A requires a large amount of the base material liquid because the projections are provided by molding the substrate into a thick structure to obtain a support shape. Further, the microneedle array is not used in a state of being stored in a container, but used by being mounted on an applicator. The microneedle array described in WO2010/140401A is a microneedle array that is integrated with the support, and the shape designed to mount the array on the container has not been examined.

The present invention has been made in consideration of the above-described circumstances, and an object thereof is to provide a method of producing a microneedle array with excellent mountability on a container and excellent handleability such as puncture or disposal, a microneedle array, and a microneedle array unit.

In order to achieve the object of the present invention, there is provided a method of producing a microneedle array, comprising: a support member placing step of placing a support member which includes a disk portion having a through-hole and a columnar portion formed on a first surface of the disk portion and extending in a direction perpendicular to the disk portion, on a pattern surface of a mold having needle-like depressions such that a second surface of the disk portion on a side opposite to the first surface faces the pattern surface; a base material liquid filling step of supplying a base material liquid from a side of the first surface of the disk portion and filling the needle-like depressions with the base material liquid; and a drying step of drying the base material liquid to integrally mold a base material layer and the support member.

In order to achieve the object of the present invention, there is provided a microneedle array according to the present invention, comprising: a sheet formed of a base material layer; a plurality of needle-like protrusions arranged on one surface of the sheet; and a support member, in which the support member includes a disk portion having a through-hole and a columnar portion formed on a first surface of the disk portion and extending in a direction perpendicular to the disk portion, a second surface of the disk portion on a side opposite to the first surface is disposed inside the base material layer, and the sheet is an integrally molded body which is obtained by molding integrally with the support member.

In order to achieve the object of the present invention, there is provided a microneedle array unit according to the present invention, comprising: the microneedle array described above; and a container which accommodates the microneedle array, in which the container includes an accommodation portion which has an opening, a deformed portion which is disposed on a side opposite to the opening and formed integrally with the accommodation portion, a binding portion which is provided in the accommodation portion of the deformed portion and bound to the columnar portion of the microneedle array, and a lid member which seals the opening, the binding portion of the container is fitted and bound to the columnar portion of the microneedle array, the deformed portion is deformed by receiving an external force in a direction of the opening and presses the microneedle array through the columnar portion, and the microneedle array is pushed out of the accommodation portion by being pressed, the deformed portion maintains a deformed state, and the microneedle array is pressed.

According to the present invention, since it is not necessary to separately perform storage of the microneedle array in a container and storage of the support member therein by integrating the microneedle array with the support member, packaging of the microneedle array performed in a sterile room can be performed in a simple step. Further, puncture of the microneedle array can be carried out in a state of being packaged in the container by fixing the support member of the microneedle array to the container so that the support member is integrated with the container. Further, the treatment can be carried out in a state where the microneedle array is integrated with the container even after the puncture and dissolution. In this manner, it is possible to easily dispose of the microneedle array and the container, prevent the microneedle array from remaining on a patient side, and improve the safety. As described above, since it is not necessary to take out the microneedle from the container and the process from puncture to disposal of the microneedle can be performed in a state where the microneedle array is integrated with the container, the handleability of the microneedle array can be improved.

### BRIEF DESCRIPTION OF THE DRAWINGS

FIG. 1 is a step view illustrating a procedure for producing a microneedle array.

FIG. 2 is a step view illustrating a procedure for producing a microneedle array.

FIG. 3 is a step view illustrating a procedure for producing a microneedle array.

FIG. 4 is a step view illustrating a procedure for producing a microneedle array.

FIG. 5 is a step view illustrating a procedure for producing a microneedle array.

FIG. 6 is a step view illustrating a procedure for producing a microneedle array.

FIG. 7 is a step view illustrating a procedure for producing a microneedle array.

FIG. 8 is a perspective view of a support member.

FIG. 9 is a perspective view of a microneedle array.

FIG. 10 is a perspective view illustrating another example of the support member.

FIG. 11 is a perspective view illustrating still another example of the support member.

FIG. 12 is a perspective view of a microneedle array unit.

FIG. 13 is a cross-sectional view of the microneedle array unit illustrated in FIG. 12.

FIG. 14 is a view for describing a step of puncturing the skin with a microneedle array.

FIG. 15 is a view for describing a step of puncturing the skin with a microneedle array.

FIG. 16 is a view for describing a step of puncturing the skin with a microneedle array.

### DESCRIPTION OF THE PREFERRED EMBODIMENTS

Hereinafter, a method of producing a microneedle array, a microneedle array, and a microneedle array unit according to the embodiment of the present invention will be described with reference to the accompanying drawings.

Method of Producing Microneedle Array

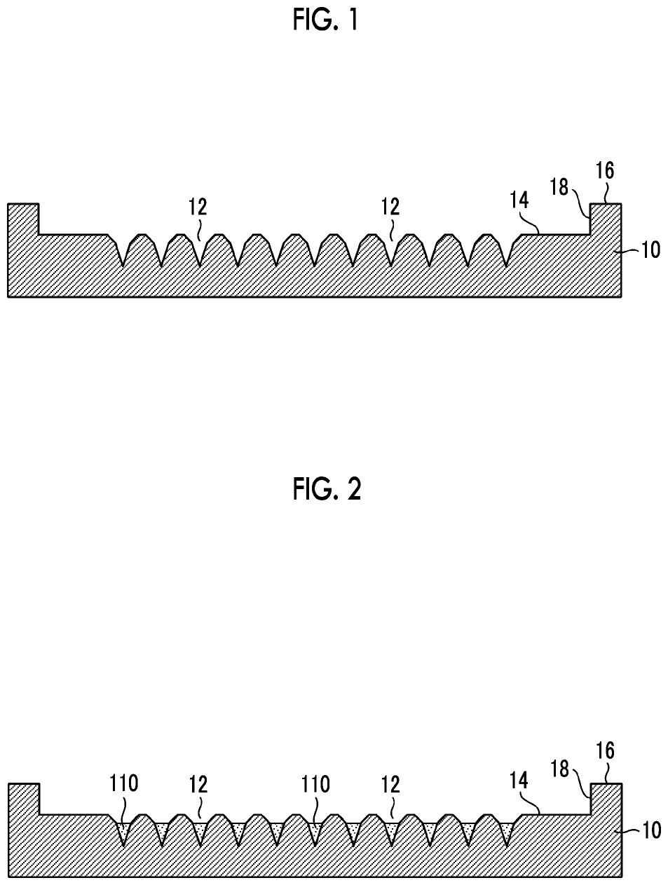

FIGS. 1 to 7 are step views illustrating a procedure for producing a microneedle array. In the production of the microneedle array, first, a mold 10 having needle-like depressions 12 is prepared as illustrated in FIG. 1. For example, the mold 10 can be produced by performing the following steps.

In production of the mold 10, a first mold is formed by performing imprint on a resin master from a precursor on which a projection pattern corresponding to needle-like protrusions of a microneedle array to be produced is formed. A duplicate mold is formed by performing an electroforming treatment after the formation of the first mold. Next, a mold sheet having needle-like depressions 12, which is a reverse type of the duplicate mold, is formed using a resin film from the duplicate mold. Finally, the mold 10 having needle-like depressions is formed by punching the mold sheet and then cutting the mold sheet for each pattern.

As the material of the mold 10, a medical grade silicone material (such as MDX-4210, manufactured by Dow Corning Corp.), a UV curable resin which is cured by irradiation with ultraviolet rays, or a plastic resin such as polystyrene or polymethyl methacrylate (PMMA) can be used.

Next, as illustrated in FIG. 2, a drug layer 110 including a drug in the needle-like depressions 12 is formed by supplying a drug solution to the needle-like depressions 12 and drying the drug solution. The drug layer 110 is formed by coating a region 14 where the needle-like depressions 12 have been formed with the drug solution including the drug. The coating method is not particularly limited and, for example, the coating can be performed by supplying the drug solution through a nozzle. Further, a spot deposition method may be used. After the supply of the drug solution, the drug solution can be sucked from the rear surface of the mold 10, and the filling of the needle-like depressions 12 with the drug solution can be accelerated.

After the needle-like depressions 12 are filled with the drug solution, the drug solution is dried to form the drug layer 110. The drug is dried by controlling the temperature and humidity conditions to optimize the drying speed so that adhesion of the drug solution to the wall surface of the needle-like depressions 12 can be reduced, and the drying can be promoted while the drug solution is collected at the tips of the needle-like depressions 12 by drying the drug solution.

By drying the drug solution, the drug solution is solidified and can be more contracted than the drug solution in a state of filling the needle-like depressions. In this manner, the drug layer 110 can be easily peeled off from the needle-like depressions 12 at the time of peeling the microneedle array 120 from the mold 10.

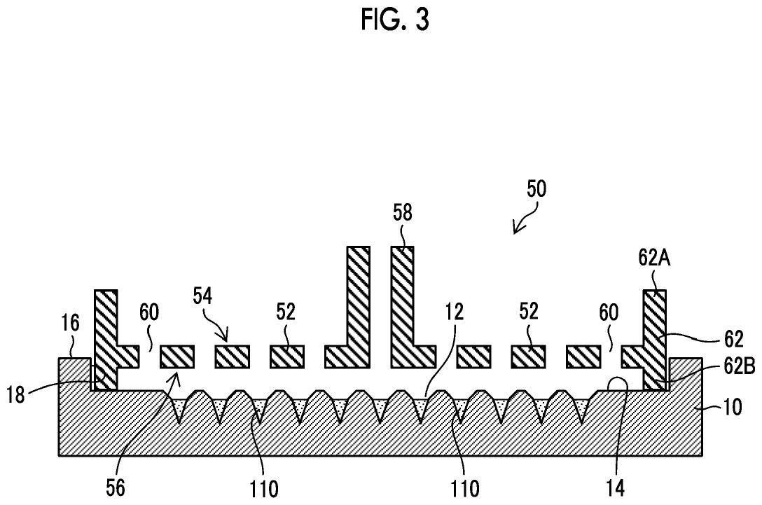

Next, as illustrated in FIG. 3, the support member 50 is placed on the region 14 (corresponding to the pattern surface) of the mold 10 where the needle-like depressions 12 have been formed (support member placing step). FIG. 8 is a perspective view of the support member 50. The support member 50 comprises a disk portion 52 having a first surface 54 and a second surface 56, and a columnar portion 58 formed on the first surface 54 of the disk portion 52 and extending in a direction perpendicular to the disk portion 52. In FIG. 8, the columnar portion 58 is formed in a hollow shape with a hollow center. The columnar portion 58 serves as a fitted portion at the time of storing the microneedle array in the container and fixing the microneedle array thereto.

The disk portion 52 has a through-hole 60 formed by a member 52A extending radially in a radial direction from the columnar portion 58 provided at the center of the disk portion 52. The through-hole 60 serves as a passage for allowing the base material liquid 102 supplied to a side of the first surface 54 to pass through the through-hole 60 and supplying the base material liquid 102 to the mold 10 on a side of the second surface 56 in a base material liquid filling step described below. It is preferable that the area ratio of the through-hole 60 to the disk portion 52, that is, (the area of the through-hole 60 to the total area of the disk portion 52 including the through-hole 60) is set to be in a range of 25% to 75%. Further, a frame 62 molded integrally with the disk portion 52 is provided on the periphery of the disk portion 52. The support member 50 illustrated in FIG. 8 includes both a first surface frame 62A protruding toward a side of the first surface 54 and a second surface frame 62B protruding toward a side of the second surface 56.

As the material constituting the support member 50, a drug product application grade cycloolefin polymer (COP) can be used. Alternatively, a resin such as polyethylene or polypropylene can be used.

Returning to FIG. 3, the support member 50 is placed in a state where the second surface 56 of the support member 50 faces the region 14 of the mold 10 where the needle-like depressions 12 have been formed. Further, it is preferable that the shape of the disk portion 52 of the support member 50 is set as a shape corresponding to the shape of the region 14 where the needle-like depressions 12 have been formed. The mold 10 has a stepped portion 16 on the periphery of the region 14 where the needle-like depressions 12 have been formed. Further, by setting the shape of the disk portion 52 of the support member 50 as a shape corresponding to the shape of the region 14 where the needle-like depressions 12 have been formed and by placing the support member 50 along the wall portion 18 of the stepped portion 16, positioning of the support member 50 to be placed can be easily performed.

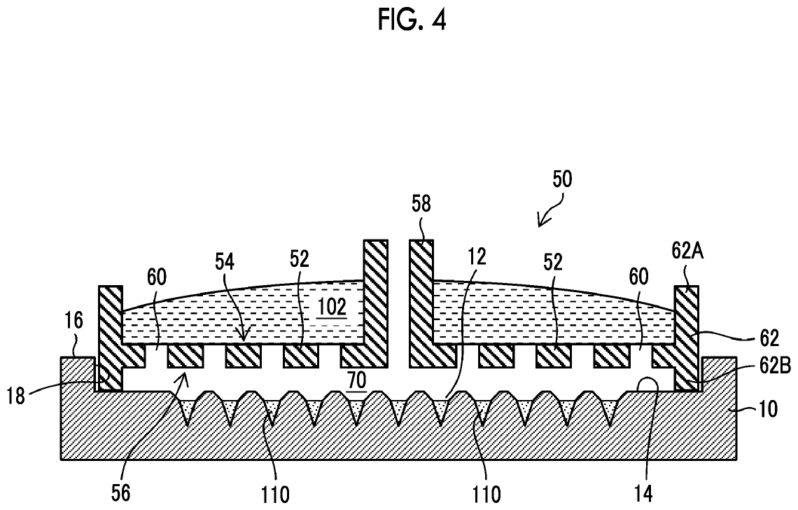

Next, as illustrated in FIG. 4, the base material liquid 102 is supplied from a side of the first surface 54 of the disk portion 52. The base material liquid 102 is a polymer-dissolved solution that forms the base material layer 112. The base material liquid 102 can be supplied by coating the region with the base material liquid using a dispenser or coating the region with the base material liquid according to a spot deposition method, but the present invention is not limited thereto. Since the drug layer 110 is solidified by being dried, diffusion of the drug contained in the drug layer 110 into the base material liquid 102 can be suppressed.

The base material liquid 102 is supplied to the inside of the first surface frame 62A of the support member 50. In this manner, leakage of the base material liquid 102 to the outside of the support member 50 can be prevented, and the shape of the base material layer 112 to be formed can be stabilized. In a case where the area of the base material layer 112 is desired to be larger than the area of the disk portion 52 of the support member 50, the base material liquid 102 may be supplied beyond the first surface frame 62A or the first surface frame 62A may not be provided.

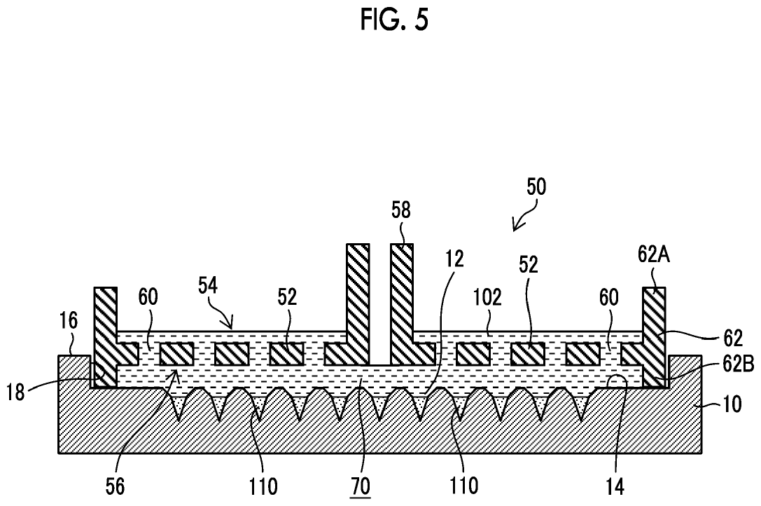

The base material liquid 102 supplied onto the first surface 54 of the support member 50 passes through the through-hole 60 of the support member 50 and moves to a side of the second surface 56 so that the needle-like depressions 12 of the mold 10 are filled with the base material liquid 102 as illustrated in FIG. 5 (base material liquid filling step). After the base material liquid 102 is supplied, vacuum suction may be performed from a side of the mold 10 opposite to the region 14 where the needle-like depressions 12 have been formed. By performing the vacuum suction, the needle-like depressions 12 can be filled with the base material liquid 102. Further, in a case where bubbles are present in the base material liquid 102, the bubbles can be removed by suction.

In addition, as illustrated in FIG. 4, a gap 70 is formed between the region 14 of the mold 10 where the needle-like depressions 12 have been formed and the second surface 56 of the disk portion 52 of the support member 50 in the height direction by providing the second surface frame 62B on the support member 50. By providing the gap 70, the base material liquid 102 that has passed through the through-hole 60 of the support member 50 can easily move the gap 70 by the surface tension. In this manner, the needle-like depressions 12 can be easily filled with the base material liquid 102. Further, in a case where it is not necessary to provide the gap 70, the needle-like depressions 12 may be filled with the base material liquid 102 in a state where the region 14 of the mold 10 where the needle-like depressions 12 have been formed is in contact with the second surface 56 of the disk portion 52 of the support member 50 without providing the second surface frame 62B.

After the needle-like depressions 12 of the mold 10 are filled with the base material liquid 102, it is preferable that the disk portion 52 of the support member 50 is in contact with the base material liquid 102 and also preferable that the disk portion 52 of the support member 50 is covered with the base material liquid 102 as illustrated in FIG. 5.

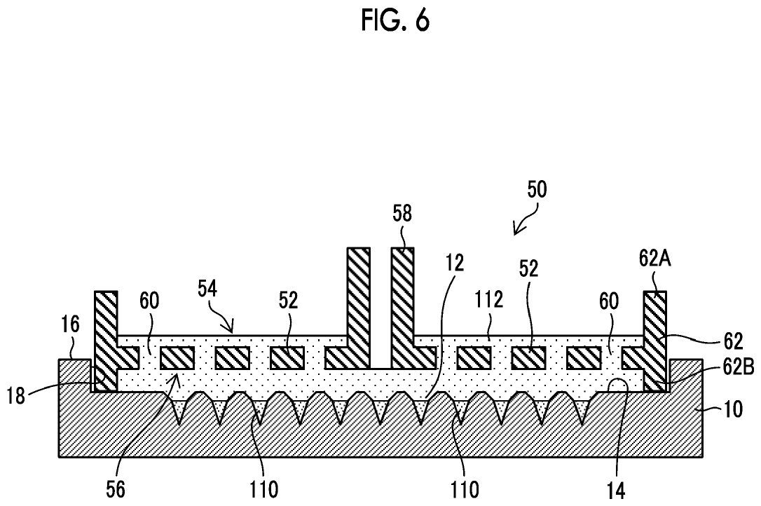

After the needle-like depressions 12 of the mold 10 are filled with the base material liquid 102, the base material liquid 102 is dried and solidified (drying step). In this manner, as illustrated in FIG. 6, the base material layer 112 can be formed on the drug layer 110 so that the microneedle array 120 having the drug layer 110 and the base material layer 112 is formed. Further, the microneedle array 120 which is an integrally molded body formed by drying and solidifying the base material liquid 102 in a state where the base material liquid 102 is in contact with the disk portion 52 so that the support member 50 is integrally molded with the base material layer 112 can be obtained. The end of the columnar portion 58 is provided so as to be exposed from a side of the base material layer 112 opposite to the surface on which the drug layer 110 is formed. In this manner, the tip thereof can be fixed to the binding portion 318 of the container 310 described below.

The moisture content of the microneedle array 120 due to the drying is set as appropriate. Further, in a case where the moisture content of the base material layer 112 becomes extremely small due to the drying, since the microneedle array becomes unlikely to be peeled off, it is preferable that the moisture content in a state where the elastic force is maintained is allowed to remain.

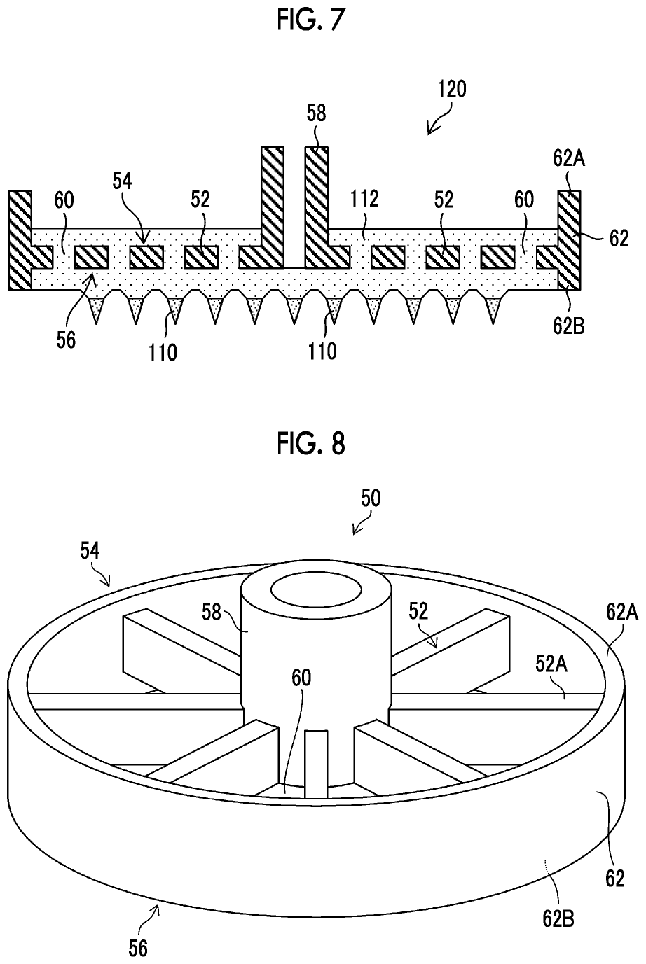

Finally, as illustrated in FIG. 7, the microneedle array 120 is produced by peeling the dried microneedle array 120 off from the mold 10.

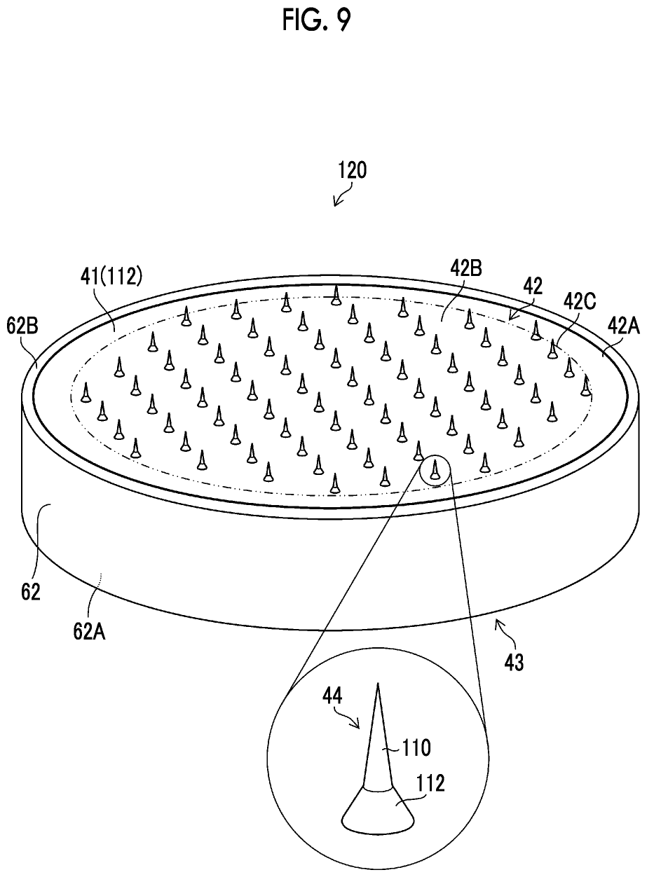

FIG. 9 is a perspective view of the microneedle array as viewed from a side of the needle-like protrusion 44. The produced microneedle array 120 comprises a sheet 41 formed of the base material layer 112, and a plurality of needle-like protrusions 44 arranged on one surface 42 of the sheet 41. In the needle-like protrusion 44, the tip thereof is formed of the drug layer 110 and the base end thereof is formed of the base material layer 112. The needle-like protrusion 44 forms a microneedle. The plurality of needle-like protrusions 44 are arranged in a microneedle region 42B inside an outer peripheral surface 42A of the one surface 42. As illustrated in FIG. 9, the boundary between the outer peripheral surface 42A and the microneedle region 42B becomes an imaginary line 42C connecting the outermost needle-like protrusions 44 among the plurality of needle-like protrusions 44.

The shapes and the dimensions of the sheet 41 and the needle-like protrusion 44 may be selected depending on the applications of the microneedle array 120. In the embodiment, the example in which the sheet 41 has a circular shape has been described, but the sheet 41 may have a rectangular shape. In addition, the shape of the disk portion 52 of the support member 50 may correspond to the shape of the sheet 41, and the shape thereof may be a rectangular shape without being limited to a circular shape.

The needle-like protrusion 44 may have a substantially conical shape, a columnar shape, a frustum shape, or the like. In the embodiment, the needle-like protrusion 44 is formed in order of a truncated cone portion and a cone from the one surface 42 toward the tip, but is not particularly limited as long as the skin can be punctured by the needle-like protrusion. It is preferable that the needle-like protrusions 44 are arranged in an array in a state where columns (lateral line) and rows (horizontal line) are uniformly spaced.

The sheet 41 of the microneedle array 120 has a diameter of, for example, 10 mm to 30 mm. Further, the needle-like protrusion 44 has a length of, for example, 0.2 mm to 1.5 mm. Further, for example, four to 1000 needle-like protrusions 44 are arranged on one surface 42 of the sheet 41. However, the present invention is not limited to these values.

The columnar portion 58 of the support member 50 is provided to protrude on a side of the other surface 43 of the microneedle array 120. The columnar portion 58 functions as a fitted portion to which the binding portion 318 provided on a deformed portion 314 of the container 310 described below is fitted.

Base Material Liquid

The base material liquid which is a solution in which a polymer resin used in the present embodiment has been dissolved will be described.

As the material of the resin polymer used in the base material liquid, it is preferable to use a resin with biocompatibility. Preferred examples of such resins include saccharides such as glucose, maltose, pullulan, chondroitin sulfate, sodium chondroitin sulfate, sodium hyaluronate, and hydroxyethyl starch; proteins such as gelatin; and biodegradable polymers such as polylactic acid and a lactic acid-glycolic acid copolymer. Although the concentration varies depending on the material, it is preferable that the concentration of the resin polymer in the base material liquid is set to be in a range of 10% by mass to 50% by mass. Further, the solvent used for dissolution may be any solvent other than warm water as long as it has volatility, and methyl ethyl ketone (MEK), alcohol, or the like can be used.

In a case where a water-soluble polymer (such as gelatin) is used, the base material liquid can be prepared by dissolving water-soluble powder in water. In a case where the water-soluble powder is unlikely to be dissolved in water, dissolution may be performed by heating water. The temperature can be appropriately selected depending on the kind of the polymer material, but it is preferable to heat water at a temperature of approximately 60° C. or lower. The viscosity of the base material liquid is preferably 2000 Pa·s or less and more preferably 1000 Pa·s or less. By appropriately adjusting the viscosity of the base material liquid, the base material liquid can be easily injected into the needle-like depressions of the mold. The viscosity of the base material liquid can be measured by, for example, a capillary viscometer, a falling ball viscometer, a rotary viscometer, or a vibration viscometer.

Drug Solution

The drug solution forming the drug layer 110 will be described. The drug solution is a solution in which the base material liquid contains a predetermined amount of drug. Whether or not the base material liquid includes a predetermined amount of drug is determined based on whether or not a drug effect can be exhibited at the time of puncturing the body surface. Therefore, the expression “including a predetermined amount of drug” indicates that the base material liquid contains the drug in an amount that enables exhibition of the drug effect at the time of puncturing the body surface.

The drug contained in the drug solution is not limited as long as the drug has a function as a drug. In particular, it is preferable that the drug is select from peptides, proteins, nucleic acids, polysaccharides, vaccines, pharmaceutical compounds belonging to water-soluble low-molecular-weight compounds, and cosmetic components.

The concentration of the polymer in the drug solution (the concentration of the polymer excluding the drug in a case where the drug itself is the polymer) is preferably in a range of 0% by mass to 30% by mass. Further, the viscosity of the drug solution is preferably 100 Pa·s or less and more preferably 10 Pa·s or less.

Other Embodiments of Support Member

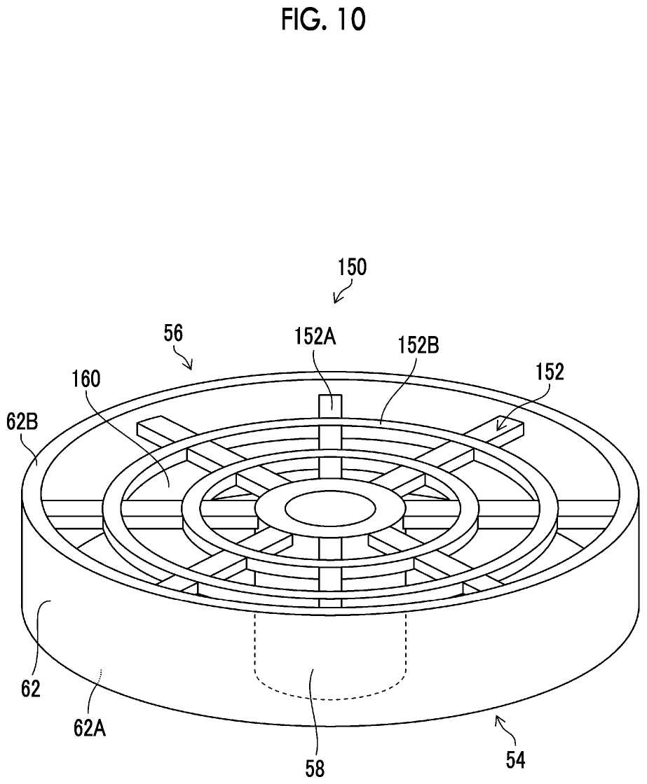

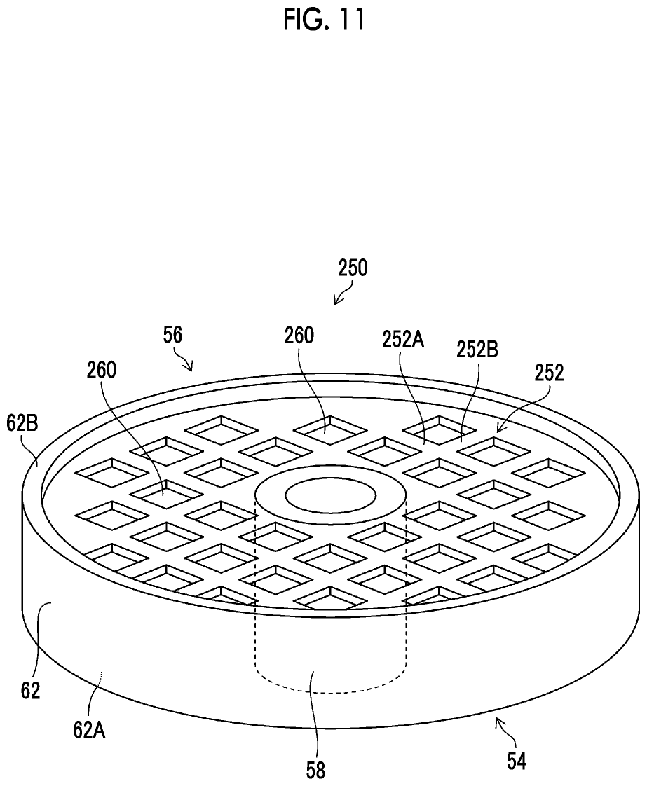

FIGS. 10 and 11 are perspective views illustrating other embodiments of the support member, as viewed from a side of the second surface. In FIGS. 10 and 11, the shape of the through-hole formed in the disk portion is different from that of the support member 50 illustrated in FIG. 7.

In a support member 150 illustrated in FIG. 10, a disk portion 152 is formed of a member 152A radially extending from the columnar portion 58 provided at the center of the disk portion 152 in the radial direction and a concentric member 152B, and a through-hole 160 is formed therebetween. That is, in the support member 150 illustrated in FIG. 10, the disk portion 152 is formed in a spider web shape. Further, in a support member 250 illustrated in FIG. 11, the disk portion 252 is formed in a mesh shape by a member 252A extending in one direction and a member 252B extending in a direction orthogonal to the one direction. A through-hole 260 is formed between the member 252A extending in one direction and the member 252B extending in a direction orthogonal to the one direction.

Even in the shape of the disk portion of the support member illustrated in FIGS. 10 and 11, the base material liquid 102 supplied to a side of the first surface 54 passes through the through-hole so that the needle-like depressions 12 of the mold 10 can be filled with the base material liquid. The shape of the disk portion is not particularly limited as long as the base material liquid 102 is allowed to pass through the through-hole and the support member can be molded integrally with the base material layer.

Microneedle Array Unit

Next, a microneedle array unit having a microneedle array will be described. The microneedle array unit has a microneedle array and a container that accommodates the microneedle array. Further, the container comprises an accommodation portion that accommodates the microneedle array, and a lid member that seals an opening provided in the accommodation portion. In the microneedle array unit, a part of the container is deformed by applying an external force from a side opposite to the opening, the microneedles are pushed out of the container, and the microneedle array is pressed by the deformed container.

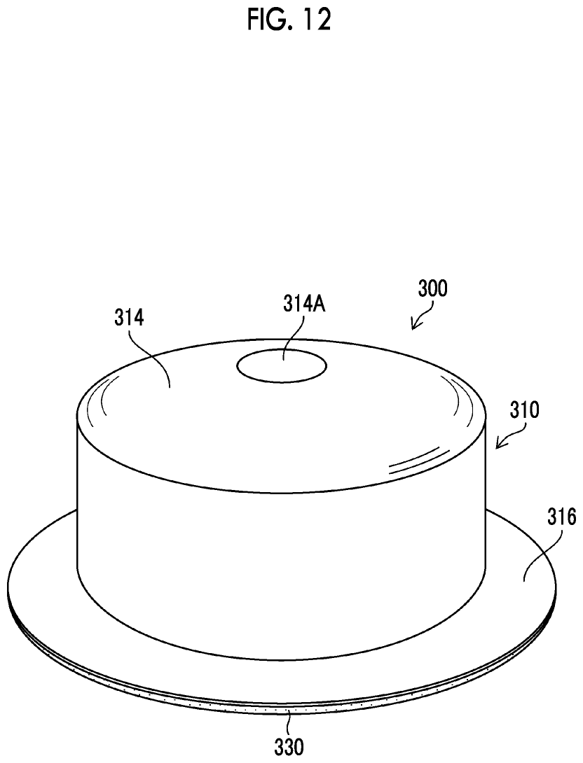

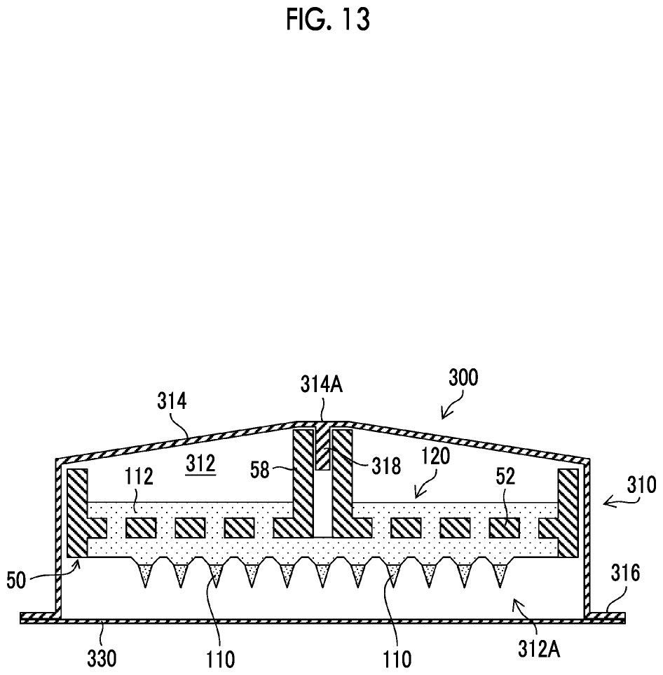

FIG. 12 is a perspective view of the microneedle array unit, and FIG. 13 is a cross-sectional view of the microneedle array unit illustrated in FIG. 12.

As illustrated in FIGS. 12 and 13, a microneedle array unit 300 includes a container 310. The container 310 includes an accommodation portion 312 for accommodating the microneedle array 120, a deformed portion 314 integrated with the accommodation portion 312, and a flange portion 316 which is integrated with the accommodation portion 312 and extends outward from the periphery of an opening 312A.

The accommodation portion 312, the deformed portion 314, and the flange portion 316 of the container 310 have a circular shape in plan view. However, the shapes of the accommodation portion 312, the deformed portion 314, and the flange portion 316 are not limited thereto. It is preferable that the shapes and the sizes of the accommodation portion 312 and the deformed portion 314 correspond to the shape and size of the microneedle array 120. The flange portion 316 is a portion that comes into contact with the skin at the time of puncturing the skin with the microneedle array 120. The flange portion 316 is provided on the entire periphery of the accommodation portion 312. The entire periphery indicates that the flange portion 316 surrounds the entire circumference of the accommodation portion 312.

As illustrated in FIG. 13, the accommodation portion 312 has an internal space defined by an inner wall and the opening 312A. The opening 312A of the accommodation portion 312 is sealed by the lid member 330. The accommodation portion 312 is sealed by bringing the periphery of the lid member 330 into contact with the flange portion 316.

The deformed portion 314 is disposed on a side opposite to the microneedle array 120 in the accommodation portion 312 with respect to the opening 312A and is integrated with the accommodation portion 312. In the embodiment, for example, the deformed portion 314 is formed in a convex shape with a vertex 314A separated from the microneedle array 120. The convex shape indicates that the vertex 314A is not positioned in the internal space of the accommodation portion 312. The term “integrated” indicates a state where the accommodation portion 312 is connected with the deformed portion 314. For example, in a case where the accommodation portion 312 is integrated with the deformed portion 314, this integration can be achieved by separately molding the accommodation portion 312 and the deformed portion 314, fitting the accommodation portion 312 and the deformed portion 314 to each other, and welding the accommodation portion and the deformed portion. In a case where the accommodation portion 312 is integrally molded with the deformed portion 314, the integration may be carried out before or after the accommodation of the microneedle array 120 in the accommodation portion 312. In the case where the accommodation portion 312 is integrated with the deformed portion 314, the integration can be realized by integrally molding the accommodation portion 312 and the deformed portion 314. However, the present invention is not limited to these methods.

The deformed portion 314 may have a frustum shape. In the embodiment, the deformed portion has a conical shape. Further, the deformed portion may have a cone shape such as a pyramid shape, and a frustum shape or a dome shape can be employed. Further, the deformed portion 314 may have, for example, an internal space, and the internal space of the deformed portion 314 can communicate with the internal space of the accommodation portion 312. The accommodation portion 312 has a structure closed by the deformed portion 314 on a side opposite to the opening 312A.

The flange portion 316 is integrated with the accommodation portion 312 and comes into contact with the skin as described below. In the embodiment, the flange portion 316 extends outward from a position of the opening 312A of the accommodation portion 312. The flange portion 316 is formed so as to be parallel to the sheet of the microneedle array 120. The concept of “parallel” includes parallel and substantially parallel. As described below, the shape of the flange portion 316 is not particularly limited as long as the flange portion comes into contact with the skin. In a case where the accommodation portion 312 is integrated with the flange portion 316, the same method as in the case where the accommodation portion 312 is integrated with the deformed portion 314 can be applied.

The binding portion 318 that is bound to the microneedle array 120 and fixes the microneedle array 120 to the container 310 is provided on a side of the accommodation portion 312 of the deformed portion 314. The microneedle array 120 is fixed to the container 310 by binding the binding portion 318 to the hollow columnar portion 58 of the microneedle array 120 so that the microneedle array 120 is integrated with the container 310. Further, the method of binding the binding portion 318 to the columnar portion 58 is not limited to a method of fitting the binding portion 318 to the hollow portion of the columnar portion 58 using the binding portion 318 as a protrusion as illustrated in FIG. 13. For example, the columnar portion can also be fixed to the binding portion by using the binding portion as a depression and fitting the columnar portion to the depression. In this case, the columnar portion 58 may not be necessarily hollow.

It is preferable that the container 310 constituting the microneedle array unit 300 is formed of, for example, a polyethylene resin, a polypropylene resin, or a mixture thereof. However, the present invention is not limited thereto. It is preferable that each of these materials satisfies the “Specification of Aqueous Injection Container made of Plastic (hereinafter, simply referred to as injection container grade)” of Japanese Pharmacopoeia. In addition, the container 310 may be formed of various other resin materials satisfying the same specification.

In particular, a material in which the shape is deformed at the time of the deformed portion 314 receiving an external force and the deformed shape is maintained is selected from among these materials. The material to be used is determined in consideration of the shape and thickness of the deformed portion 314, the magnitude of the external force required for deformation, and the like.

According to the microneedle array 120 of the present embodiment, packaging of the microneedle array 120 in a sterile room can be easily performed by integrally molding the support member 50 with the sheet 41 (base material layer 112). Since the microneedle array 120 is used by puncturing the skin, it is necessary to protect the microneedles until the skin is punctured. Further, in order to ensure the sterility of the microneedle array 120, the packaging of the microneedle array in the container 310 is performed in a sterile room, and the microneedle array is stored in the container 310 until immediately before use. In a case where the microneedle array 120 is integrated with the support member 50, the container 310, the support member 50, and the microneedle array 120 are separately fixed in a sterile room. Therefore, it takes time to work in a sterile room. Further, members for fixing the support member 50 and the microneedle array 120 are also required. By integrally molding the support member 50 with the sheet 41 and by fixing the support member 50 to the container 310, the microneedle array 120 can be fixed to the container 310, and the packaging step in a sterile room can be simplified. Further, the number of members for fixing the support member 50 and the microneedle array 120 can be reduced.

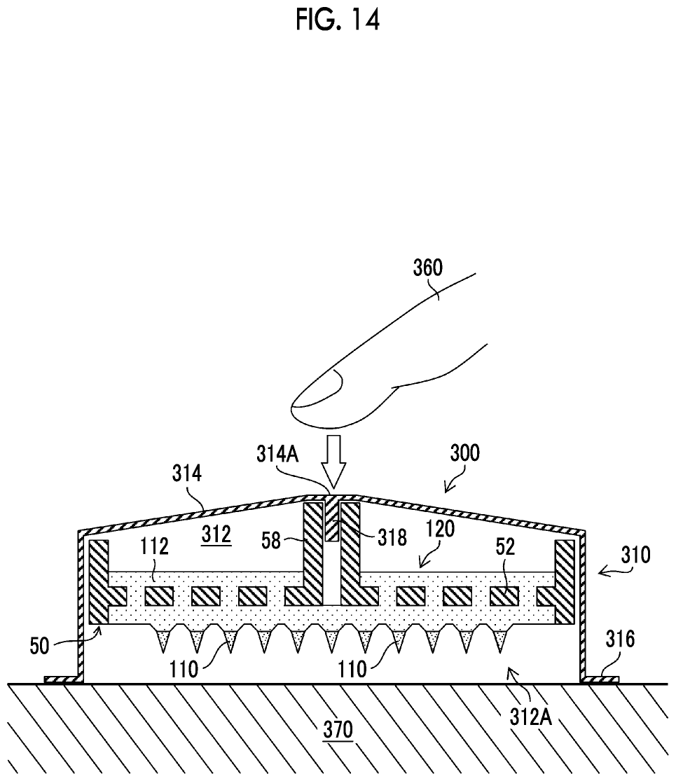

Next, a step of puncturing the skin with the microneedle array 120 using the microneedle array unit 300 will be described with reference to FIGS. 14 to 16. FIGS. 14 to 16 are cross-sectional views of the microneedle array unit 300 illustrating the step of puncturing the skin with the microneedle array 120.

First, the lid member 330 that seals the opening 312A of the accommodation portion 312 is peeled off from the container 310. The needle-like protrusions 44 of the microneedle array 120 are protected from damage because of the lid member 330. It is preferable that the lid member 330 has a knob in order to facilitate the peeling.

Next, the container 310 is positioned on a skin 370 as illustrated in FIG. 14. The opening 312A of the accommodation portion 312 is positioned toward the skin 370, and the needle-like protrusions 44 of the microneedle array 120 are oriented toward the skin 370. The flange portion 316 extending outward from the accommodation portion 312 is brought into contact with the skin 370. A finger 360 is positioned at a position separated from the deformed portion 314 in order to apply an external force to the deformed portion 314 in a direction of the opening 312A. The microneedle array 120 is supported by fitting the binding portion 318 of the container 310 and the columnar portion 58 of the support member 50 and is positioned in the internal space of the accommodation portion 312.

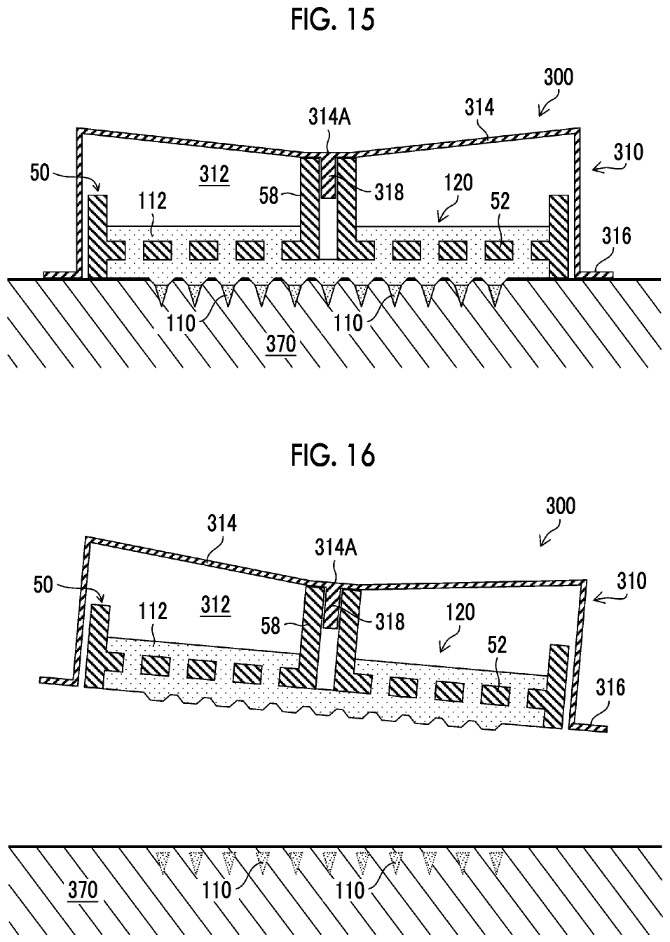

After the container 310 is positioned on the skin 370, the deformed portion 314 is pressed toward the skin 370 by the finger 360. The deformed portion 314 is deformed by receiving an external force in a direction of the opening 312A. As illustrated in FIG. 15, the deformed portion 314 is deformed by an external force, and the deformed shape of the deformed portion 314 is maintained even after the external force is removed. The deformed portion 314 which has been deformed presses the microneedle array 120 toward the skin 370.

As described above, the microneedle array 120 is fixed to the container 310 by fitting the binding portion 318 provided on the deformed portion 314 and the columnar portion 58 provided on the support member 50. Therefore, by pressing the deformed portion 314, the microneedle array 120 is pushed out of the accommodation portion 312 through the columnar portion 58 in a state where the microneedle array 120 is fixed to the container 310. The microneedle array 120 passes through the opening 312A, and the needle-like protrusions 44 of the microneedle array 120 puncture the skin 370.

After the puncture, since the microneedle array 120 is pressed by the deformed portion 314 of the container 310 until the drug of the microneedle array 120 is administered, falling of the microneedle array 120 off the skin 370 without pressing of the finger 360 is prevented.

By designing the outer diameter of the microneedle array 120, that is, the outer diameter of a frame 62 of the support member 50 to be slightly smaller than the inner diameter of the accommodation portion 312, it is possible to prevent the pressed microneedle array 120 from being greatly deviated from a direction of the opening 312A. Therefore, the skin 370 can be vertically punctured by the needle-like protrusions 44 of the microneedle array 120.

Finally, the microneedle array 120 is peeled off together with the container 310 as illustrated in FIG. 16. The peeling of the microneedle array is performed after the skin 370 is punctured by the needle-like protrusions 44 of the microneedle array 120 and the time for which the drug layer 110 forming the needle-like protrusions 44 remains in the skin is elapsed. In this manner, the drug can be injected into the skin. Since the microneedle array 120 is fixed to and integrated with the container 310, the treatment can be performed without separating the microneedle array 120 and the container 310 from each other even at the time of puncturing the skin and peeling the microneedle array from the skin. Therefore, separate disposal of the microneedle array 120 and the container 310 is not required at the time of puncturing the skin with the microneedle array 120, and the disposal can be easily carried out. Further, by disposing of the microneedle array 120 and the container 310 together, it is possible to prevent the microneedle array 120 from remaining on a patient side and improve the safety for the patient. In this manner, by fixing the microneedle array 120 to the container 310 so that the microneedle array 120 is integrated with the container 310, a microneedle array unit with improved handleability from the puncturing the skin to the disposal of the microneedle array 120 can be obtained.

### Example

The excised pig skin was punctured by microneedles using a microneedle array unit produced according to the following method, and the puncture properties and separation of the microneedle array from the container were confirmed.

The support member 50 illustrated in FIG. 8 which was formed by using a biocompatible resin was placed in the region 14 of the mold 10 formed of silicone rubber where the needle-like depressions 12 had been formed such that the second surface 56 of the support member 50 faced the region. A 40% aqueous solution of sodium chondroitin sulfate was supplied as the base material liquid 102 from the first surface 54 of the support member 50, and the needle-like depressions 12 of the mold 10 were filled with the solution. In this example, in order to confirm the puncture properties of the microneedle and the separation of the microneedle array from the container, the drug layer was not formed, and the microneedle array was produced using only the base material liquid.

The microneedle array 120 integrated with the support member 50 was obtained by filling the needle-like depressions 12 of the mold 10 with the base material liquid 102 and then drying the base material liquid. The obtained microneedle array 120 was put into the container 310 having the binding portion 318 (protrusion) illustrated in FIG. 13, the columnar portion 58 of the support member 50 and the binding portion 318 of the container 310 were fitted to each other, and the microneedle array 120 was fixed to the container 310. The microneedle array unit 300 was produced by sealing the opening 312A of the container 310 with a sealing member as the lid member 330.

The sealing member was peeled off, the opening 312A of the container 310 was disposed toward the excised pig skin, and the rear surface side (the deformed portion 314 side) of the container 310 was pressed with a thumb to puncture the excised pig skin with microneedles. After elapse of a predetermined time from the puncture, the microneedle array 120 and the container 310 were simultaneously peeled off. The puncture of the microneedles was able to be carried out without any problem. In addition, the process from the puncture of the microneedle to the peeling of the microneedle array 120 and the container 310 was able to be performed without separation of the microneedle array 120 and the container 310.

### EXPLANATION OF REFERENCES

10: mold

12: needle-like depression

14: region where needle-like depressions have been formed

16: stepped portion

18: wall portion

41: sheet

42: one surface

42A: outer peripheral surface

42B: Microneedle region

42C: imaginary line

43: other surface

44: needle-like protrusion

50, 150, 250: support member

52, 152, 252: disk portion

52A, 152A, 152B, 252A, 252B: member

54: first surface

56: second surface

58 columnar portion

60, 260: through-hole

62: frame

62A: first surface frame

62B: Second surface frame

70: gap

102: base material liquid

110: drug layer

112: base material layer

120: microneedle array

300: microneedle array unit

310: container

312: accommodation portion

312A: opening

314: deformed portion

314A: vertex

316: flange portion

318: binding portion

330: lid member

360: finger

370: skin

What is claimed is:

1. A method of producing a microneedle array, comprising:
a support member placing step of placing a support member which includes a disk portion having a through-hole and a columnar portion formed on a first surface of the disk portion and extending in a direction perpendicular to the disk portion, on a pattern surface of a mold having needle-like depressions such that a second surface of the disk portion on a side opposite to the first surface faces the pattern surface;
a base material liquid filling step of supplying a base material liquid from a side of the first surface of the disk portion and filling the needle-like depressions with the base material liquid; and
a drying step of drying the base material liquid to integrally mold a base material layer and the support member.

2. The method of producing a microneedle array according to claim 1,
wherein vacuum suction is performed from a surface of the mold on a side opposite to the pattern surface in the base material liquid filling step.

3. The method of producing a microneedle array according to claim 1,
wherein a gap is provided between the pattern surface of the mold and the second surface of the support member in the support member placing step.

4. The method of producing a microneedle array according to claim 1,
wherein a ratio of an area of the through-hole to an area of the disk portion is in a range of 25% to 75%.

5. The method of producing a microneedle array according to claim 1,
wherein a drug layer including a drug is formed in the needle-like depressions before the support member placing step.

6. A microneedle array comprising:
a sheet formed of a base material layer;
a plurality of needle-like protrusions arranged on one surface of the sheet; and
a support member,
wherein the support member includes a disk portion having a through-hole and a columnar portion formed on a first surface of the disk portion and extending in a direction perpendicular to the disk portion,
a second surface of the disk portion on a side opposite to the first surface is disposed inside the base material layer, and
the sheet is an integrally molded body which is obtained by molding integrally with the support member.

7. The microneedle array according to claim 6,
wherein an end of the columnar portion is exposed from other surface of the sheet on a side opposite to the one surface.

8. The microneedle array according to claim 6,
wherein a ratio of an area of the through-hole to an area of the disk portion is in a range of 25% to 75%.

9. The microneedle array according to claim 6, further comprising:
a drug layer which includes a drug at tips of the needle-like protrusions.

10. A microneedle array unit comprising:
the microneedle array according to claim 6; and
a container which accommodates the microneedle array,
wherein the container includes
an accommodation portion which has an opening,
a deformed portion which is disposed on a side opposite to the opening and formed integrally with the accommodation portion,
a binding portion which is provided in the accommodation portion of the deformed portion and bound to the columnar portion of the microneedle array, and
a lid member which seals the opening,
the binding portion of the container is fitted and bound to the columnar portion of the microneedle array,
the deformed portion is deformed by receiving an external force in a direction of the opening and presses the microneedle array through the columnar portion, and
the microneedle array is pushed out of the accommodation portion by being pressed, the deformed portion maintains a deformed state, and the microneedle array is pressed.
